(19) United States
(12) Patent Application Publication
(10) Pub. No.: US 2023/0398256 A1
(43) Pub. Date: Dec. 14, 2023

(54) AN INTEGRATED AIR, LIQUID AND SURFACE STERILIZATION METHOD AND SYSTEM THEREOF

(71) Applicant: PERSAPIEN INNOVATIONS PRIVATE LIMITED, Delhi North West (IN)

(72) Inventors: Shashi RANJAN, Aurangabad (IN); Debayan SAHA, Kolkata (IN)

(21) Appl. No.: 18/032,223

(22) PCT Filed: Apr. 19, 2022
(86) PCT No.: PCT/IB2022/053630

§ 371 Date: Apr. 17, 2023

(30) Foreign Application Priority Data
Apr. 20, 2021 (IN) 202111018226

(51) Int. Cl. A61L 9/22; A61L 2/03; A61L 9/14

(52) U.S. Cl. CPC A61L 9/22; A61L 2/035; A61L 9/14; A61L 2209/213

(57) ABSTRACT

The present invention discloses an integrated technique for sterilization or disinfection or sanitization, to remove microbial load in air or in liquid or on solid surfaces using water, gases and electrical energy. Electrical energy is used in the present invention to generate ions or electrically activated species from mixture of gases generated from electrolysis, air and water mist which can be directly used for sterilization or can be dissolved in water to create electrically activated water. This water is directly used for surface disinfection or may be converted into activated water mist for disinfection of air. The gas plasma or activated water mist may also be dissolved in water for disinfection of water.

### FIELD OF INVENTION

The present invention generally relates to a technique for sterilization, disinfection/decontamination or sanitization, to remove microbial load in air in a given space or in liquid or on solid surfaces. The present invention involves the removal or inactivation of microbes such as bacteria, viruses, fungus or any other microbes suspended in air or present inside liquid or settled on solid surfaces in an integrated way.

### BACKGROUND OF THE INVENTION

The recent outbreak of COVID-19 has put a big question in terms of our technical developments to solve this problem. It became a pandemic in just a few months' time. World is not experiencing viral outbreak for the first time, though not at this scale. However, we have not been able to develop optimal technologies to control such outbreak. Moreover, mankind has been grappling with the problem of microbial contamination since the start of the civilization. The best way to contain infection is to prevent it from spreading. Thus, the need for an effective disinfection technique is evident.

JP2019523038A discloses a sterilization, disinfection, sanitization or decontamination system having a chamber defining an area and a generator and/or vaporizer to generate free radical effluents having reactive oxygen, nitrogen and other species. The system requires usage of bleach for performing the method of sterilization, disinfection, sanitization or decontamination.

The current methods of sanitization involve usage of chemicals such as bleach which are harmful to human and environment. Moreover, their availability during time of crisis and lockdowns is generally questionable. Also, logistic is a big problem during emergency situation. All these factors limit the use of chemical disinfectants. Other methods use Ultra-violet light or other ionizing radiations which are harmful to health.

The present invention provides safe and chemical-free method of disinfection. It provides ways to use mostly water, air/gases and electrical energy to create antimicrobial agent as required.

### Objective of the Invention

The primary objective of the present invention is development of sterilization or disinfection or sanitization technique to remove microbial load in air or in liquid or on solid surfaces or in all these forms using water, air/gases and electrical energy.

Yet another objective of the present invention is to provide a method of generation of electrically activated water by dissolving ions or electrically activated species generated in a gas obtained from electrolysis, air or water mist or their mixture

Yet another objective of the present invention is to generate electrically activated water mist comprising micro/nano droplets of electrically activated water.

Yet another objective of the present invention is to develop method for transport of the activated water mist from place of generation to place of required action to achieve sterilization of larger space.

Yet another objective of the present invention is to develop an integrated system for sterilization or disinfection of air, liquid, water and solid surfaces to achieve overall infection control technique.

Other objectives and advantages of the present invention will become apparent from the following description taken in connection with the accompanying drawings, wherein, by way of illustration and example, the aspects of the present invention are disclosed.

### SUMMARY OF THE INVENTION

The present invention relates to a method and system for development of a sterilization or disinfection or sanitization technique to remove microbial load in air or in liquid or on solid surfaces or in all these three forms using water, air/gases and electrical energy. The technique can be used as an independent method or an integrated module for disinfection of either air, liquid or solid or all of these three forms. The electrical energy is used in this invention to generate electrically activated species such as ions, radicals etc. by creating electric field or plasma of air/gases/mist. These electrically activated species can either be dissolved in water or is transferred from air to water droplets to create electrically activated water or electrically activated water mist. Such water can be used directly for surface disinfection and can also be converted into the activated mist which can be used for disinfection of air. For disinfection of liquid, the electrically activated species generated through the air/gas/mist plasma or through activated water mist can be dissolved in liquid/water for disinfection of the liquid/water.

### BRIEF DESCRIPTION OF DRAWINGS

The present invention will be better understood after reading the following detailed description of the presently preferred aspects thereof with reference to the appended drawings, in which the features, other aspects and advantages of certain exemplary embodiments of the invention will be more apparent from the accompanying drawings in which:

FIG. 1 illustrates the working methodology of the present invention for sterilization or decontamination or sanitization by electrically activated water and water mist.

Corresponding reference characters indicate corresponding parts throughout several views. The components in the Figure are not necessarily to scale, emphasis instead being placed upon illustrating the principals of the invention. The exemplification set out herein illustrates embodiments of the invention, and such exemplification is not to be construed as limiting the scope of the invention in any manner.

### DETAILED DESCRIPTION OF THE INVENTION

The following description describes various features and functions of the disclosed system and methods with reference to the accompanying figures. In the figures, similar symbols identify similar components, unless context dictates otherwise. The illustrative aspects described herein are not meant to be limiting. It may be readily understood that certain aspects of the disclosed system, method and apparatus can be arranged and combined in a wide variety of different configurations, all of which may not necessarily have been described herein, but should be considered part of the invention disclosed hereinafter.

These and other features and advantages of the present invention may be incorporated into certain embodiments of the invention and will become more fully apparent from the following description or may be learned by the practice of the invention as set forth hereinafter. Accordingly, those of ordinary skill in the art will recognize that various changes and modifications of the embodiments described herein can be made without departing from the scope of the invention. In addition, descriptions of well-known functions and constructions are omitted for clarity and conciseness.

The terms and words used in the following description are not limited to the bibliographical meanings, but, are merely used to enable a clear and consistent understanding of the invention. Accordingly, it should be apparent to those skilled in the art that the following description of exemplary embodiments of the present invention are provided for illustration purpose only and not for the purpose of limiting the invention.

It is to be understood that the singular forms “a”, “an” and “the” include plural referents unless the context clearly dictates otherwise.

It should be emphasized that the term “comprises/comprising” when used in this specification is taken to specify the presence of stated features, integers, steps or components but does not preclude the presence or addition of one or more other features, integers, steps, components or groups thereof.

The main aspect of the present invention relates to a method and system for sterilization or disinfection/decontamination of air, liquid water and solid surfaces by using gases/air, water and electrical energy. In a preferred embodiment, the present invention generates electrically activated water and/or electrically activated water mist. In first step, gases are produced by electrolysis of water which is used directly or is mixed with air or water mist and is exposed to electric field for generation of ions or other electrically activated species. These species are dissolved in water to obtain electrically activated water. The electrically activated water can be used for disinfection of surface. The electrically activated water can also be converted into electrically activated water mist which can be used for disinfection of larger spaces and spaces located at a distance from the place of generation of the said mist.

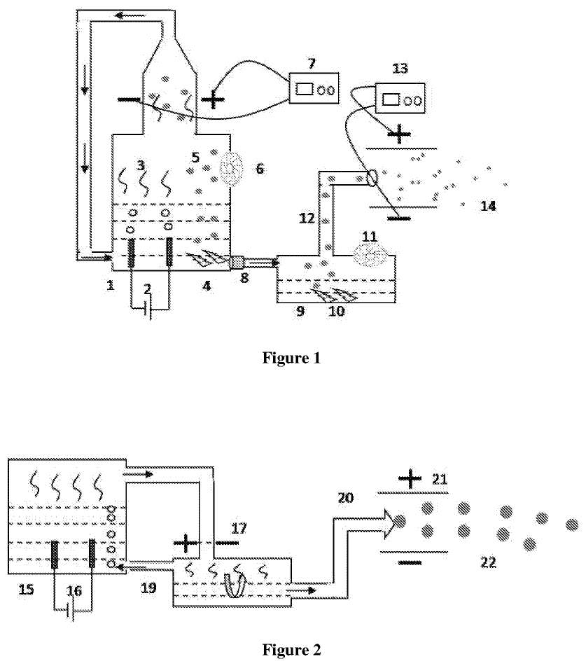

FIG. 1 of the present invention illustrates the working methodology for sterilization of a substance/space by electrically activated water and water mist. As per the preferred embodiment of the present invention, microbial load is removed by electrolyzing or hydrolyzing water in a chamber (1) in presence or absence of electrolytes, by using electrodes with DC power supply (2) to produce gases such as oxygen, hydrogen, or other gases depending on electrolytes (3). Such produced gases may be mixed with air or the water mist (5) which can be generated through ultrasonic mist maker (4). The mixing is accomplished by using a fan (6), which also propels the said mixture for the next step. The mixture is then exposed to an electric field, generally high voltage electric field generated by a power source (7) that may generate plasma using high voltage DC or AC supply with high voltage and high frequency in electrolysis chamber (1) or charging chamber (9). The generation of plasma or exposure to high voltage electric field converts gases into ionized species or generates other electrically active species such as radicals. The said ionized gas mixture or the mist is re-circulated and dissolved in the water in chamber (1) to generate activated water. The said water may be used for sterilization of surfaces by taking it out through a water pump (8) or can be transferred to charging chamber (9). The activated water may directly be used for surface disinfection or may be converted into water mist by shearing water into smaller droplets through a dispersing unit, such as but not limited to micro-droplets by using either fine nozzles or ultrasonic probes (10) or some other technique. The micro-droplets are then mixed with air using a fan (11) to produce water mist (12) which is subjected to high voltage electric field or electron clouds or ionized space generated by a power source (13) to gain charge or ions for further activation. The ions or electrical species are produced in air as well as in water droplets due to electric field. The electrical species generated in air is transferred to water droplets to allow droplets to gain charge. The micro droplets of water breaks into nano-droplets under effect of high electric repulsion within a droplet (14). These highly activated nano-droplets are then dispersed in air or is transported to other space farther from the site of generation of the said mist for continuous disinfection of air.

FIG. 2 of the present invention illustrates the working mechanism in an alternate embodiment. As per this embodiment, water is electrolyzed in first chamber (15) using electrodes connected with a power source (16) to produce gases. The produced gas mixes with air and is exposed to high power electric field (17) to generate plasma of the gas-air mixture. The plasma generates ions and other electrically active species which are dissolved in the water in second chamber (18) by recirculation and is also transported back through a connection (19) between first chamber (15) and second chamber (18). The process is run as a cyclical process to obtain electrically activated water in second chamber (18) which can be used for surface disinfection and the water is sterilized too. The activated water in second chamber (18) is converted to mist by ultrasonic transducer or fine nozzle (20) and is passed through a high power electric field (21) to generated electrically activated water mist (22) which can be used as an air disinfectant.

In an embodiment, the present invention provides a method of sterilization to remove microbial load comprises of following steps:
i. electrolyzing/hydrolyzing water in a chamber in presence or absence of at least one electrolyte to produce mixture of gases through at least one electrode with DC power supply;
ii. mixing gases obtained in step (i) with air or water mist to obtain a gas mixture;
iii. exposing the gas mixture obtained in step (ii) to an electric field usually high voltage or high power electric field generated using high voltage DC or AC supply with high voltage and high frequency in the charging chamber (1) or charging chamber (9), converting gases into ionized species and/or generating other electrically active species (such as radicals), wherein the ionized gas mixture or activated mixture can be used for sterilization of surfaces or air;
iv. dissolving the activated mixture obtained in step (iii) in water to obtain electrically activated water, thereby sterilizing the water;
v. converting the activated water obtained in step (iv) into water mist by breaking the activated water into small water droplets;
vi. mixing the water droplets obtained in step (v) with air, producing water mist;
vii. passing the water mist obtained in step (vi) through high electric field to gain charge/ion for further activation;
viii. forming electrical species/ions in air and water droplets in the presence of electric field;
ix. transferring the electrical species generated in step (vii) to water droplet, allowing the water droplets to gain charge;
x. breaking micro-droplets of water into nano-droplets through electron clouds;
xi. dispersing the electrically activated nano-droplets obtained in step (x) in air or transporting the nano-droplets in space away from the site of generation of the said mist for continuous disinfection of air.

The activated water mist is safe for human exposure and may be used to control the transmission of infection from one host to another. The water on dispersion in air as activated water mist disinfects air. The water droplets eventually settles on the surfaces, thereby decontaminating the surfaces.

The other aspect of the present invention is development of an reinforcement system where gases generated by electrolysis or hydrolysis is used for generation of electrical species or plasma to electrically activate water and electrically activated water reinforces electrolysis or hydrolysis of water. The two processes can be done simultaneously or discreetly. The two process can either be separated by space (by putting in different chamber) or by time (by conducting at different time). The electrically activated water can be converted into electrically activated mist to disinfect air and surfaces which can be remixed in the water to further increase charge density or electrical activation of the water.

As per a preferred embodiment of the present invention, the system for obtaining any one of: activated water or activated mist includes: an electrolysis chamber (1) having a pair of electrodes and a power source (2) for electrolysis of water for obtaining a plurality of gases through electrolysis (3); a first ultrasonic transducer (4) for breaking water provided in the electrolysis chamber (1) into plurality of droplets; a first fan (6) mounted on the electrolysis chamber (1) for mixing the plurality of gases (3) with air or water mist (5); the first pair of electrodes (7) for exposing the gas mixture comprising gases (3), air and water mist (5) to high power electric field; charging chamber (9) for electrical activation of water received from the electrolysis chamber (1); a second fan (11) mounted on the charging chamber configured for mixing air with water droplets so as to generate activated mist (12); a second ultrasonic transducer (10) configured for breaking activated water into smaller droplets; at least one water pump (8) for circulation of fluids between the electrolysis chamber (1) and the charging chamber (9); and a pair of second electrodes (13) connected to the charging chamber. As per this embodiment, the pair of first electrodes (7) and the pair of second electrodes (13), connected to their respective power sources is configured to generate electric field that breaks water droplet into ultrafine droplets so as to create electrically activated water mist (14).

Yet another aspect of the present invention is to disclose methods of dissolving electrical species or ionized species generated in the gas phase into bulk water or water droplets through repeated bubbling of the gas phase in water and increasing the residence time of gases in water through speed control and increasing the column length The mixing is further improved by breaking the bubbles smaller by using nano nozzles or shearing of bubbles through ultrasonic means.

Another aspect of the present invention is to develop an integrated system for sterilization or disinfection of air, liquid water and solid surfaces or things. The generation of electrical species through high voltage exposure or plasma generation of mixture of gases produced from electrolysis, air and/or mist can be used for disinfection of solid surfaces via direct and immediate exposure of such surfaces or things. The electrical activation of water can be used sterilization of water or other liquids as well as electrical activated water can be used as disinfectant. The Activated Water Mist generated from electrically activated water as mentioned before can be used for sterilization or disinfection of air as well as surfaces at a distance away from the source of generation of such mist. Thus, an integrated system for sterilization is invented here.

Another important aspect of the present invention is to develop a method for inactivation of microbes of different sizes through activated water mist which comprise of micro to nano sized water activated water droplets. The aerodynamic size of such droplet is modulated in this invention to match the aerodynamic size of targeted microbes to increase the probability to their interaction with the microbes. Such activated mist is generated by shearing electrically activated water into smaller droplets and mixing them with certain amount of air to obtain certain mist density. Such mist is them exposed to high voltage electric field of certain magnitude through certain configuration of positive and negative electrodes. Modulation of mist density, flow parameters and electrical parameters allows modulation of size of water droplets in the electrically activated mist. In this invention, a range of sizes of water droplets is generated to inactivate different microbes like virus, bacteria etc., of different sizes present in air.

Yet another aspect of the invention is to bias the system between aerosol and surface disinfection based on the modulation of size distribution of the activated water mist Smaller sizes of water droplets favors aerosol disinfection while larger sizes of droplets favors surface disinfection.

The invention also discloses a method of transmission of electrical species generated in the air from air to water droplets. The transfer of electrical species from air to water droplet is facilitate through flow based mixing of air and water droplets, through acceleration of charges by application of electric force and by the application of electromagnetic wave.

The invention discloses a method for transporting the mist over large distance through a duct or other such arrangement with minimal loss of water droplet due to coalition or precipitation by at walls of such a duct system. Electric field is applied in direction of flow of the mist particles to accelerate them to minimize coalition. A streamlined air envelop is created around the mist flow to minimize contact of the mist particles with the walls of the duct. Such air envelop also protects the water particles at turns in the duct system. Moreover, the gain of charge by water droplets is maximized in the mist which allow water particles to break down into ultrafine droplets and behave like gas which further decreases their chances of loss due to settlement at immediate surfaces.

The technology as depicted in the drawing consists of electrolysis or hydrolysis chamber where electrolysis of electrolyte solution is carried out by passing electric current through the solution. The voltage is maintained through a battery or any other power source. Generally, DC voltage is used in range of 0.01V-1000V or some other range or value. The current can be 0.01 amp-1000 amp or some other range or value. Different type of gases are produced during electrolysis including oxygen and hydrogen. The time of electrolysis is determined by the volume of electrolysis chamber and rate of flow of electrolyte solution.

The electrolyzed gas either independently or as a mixture of such gases with air or water mist is exposed to an electric field, usually high voltage or high power electric field generated by a high voltage DC generator or a high voltage high frequency AC generator, where high voltage can be the voltage more than 250V and high frequency can be more than 500 Hz, to generate electrical species or to generate plasma i.e. ionized form of such gases or such mixture. Different configuration or methods of plasma generation such as Pin-jet, Dielectric Barrier Discharge (DBD) can be used in isolation or in combination to achieve desired results. The electrical species generated through exposure to high voltage electric field or through Plasma generation is dissolved in water to electrically activate the water. The said activated water is then broken into micro-droplets in the electrolysis chamber (1) or charging chamber (9) using nozzle or ultrasonic transducers. Such water droplets are mixed in air to form mist which is then exposed to high voltage Direct Current (DC) or Alternating Current (AC) electric field further activating the water droplets through generation of electrical species in water droplets and through transfer of electrical species from air to water droplets. Water droplets in form of the mist coming out of this system is further broken down into micro or nano droplet due to Rayleigh effect. These charged droplet can be repeatedly passed through high electric field to accumulate more charge in each droplet. Different types of configuration such as needle-based, Pin-ring or conductive brush can be used for this purpose in isolation or in combination.

In an embodiment, the electrically treated gases, air, mist or their mixture in any combination is mixed through water in electrolysis chamber, through bubbling it through the water or by precipitating the mist into water droplets and then allowing it to mix with the bulk water in the said chamber. Ultrasonic waves can be used to break the gas bubble and enhance mixing.

The sterilized as well as electrically activated water stored in chamber may be used as sterilizer for different applications or for sanitization purposes.

The invention disclosed here allow generation of highly charged water droplets of size in micro and nano meter. Moreover, these droplets contain super oxides which makes the droplets very potent antimicrobial agent. Different configuration and methods can be used in isolation or in combination. This technology is very appropriate as sterilization or decontamination tool and can be used as sanitization method for air, water and surfaces simultaneously in an integrated way.

In one of the aspect, the invention discloses way to disperse activated water droplets to large volume using electrometric or physical wave. It can be obtained by high voltage high frequency power source or sonic waves.

In another aspect, the invention discloses way to disperse activated water droplets to large volume by creating air bubbles enveloping the activated water mist. The air and the mist acts, like two gas phase wherein the two gas remains separated. The said air bubble provides protection to the said mist and allow dispersion in the large volume space.

The technology disclosed in this invention can be used for different needs and in different embodiments such as Indoor spaces, outdoor spaces, human beings, cargo and hand sanitization.

In an alternate embodiment of the present invention, the system may be configured as an indoor disinfection system for air and surface disinfection in large spaces by integrating the disinfection system to HVAC or Air Handling Units (AHUs). As per this embodiment, the activated water mist returning to the AHU can be precipitated by using an electrically charged negative and positive plates. Such precipitation is achieved due to electrical charge contained in the mist droplets. The water droplets in the mist may have net positive or net negative charge which can allow their precipitation on the plate with opposite charge. The plates are arranged in-series with alternate polarity i.e. negative plate followed by positive plate and son on. The precipitated water provided in the AHU is further dissolved in the bulk water (in water storage chamber) to increase the ionization and activation of water. The water in the mist can be obtained by other process of condensation/capture. The condensation may be achieved through mechanical or electrical means, whereas capture can be facilitated by hygroscopic compounds or any refrigeration system. Activated Water Mist may be used for disinfection of larger spaces which would return to AHU system where condensation or capture of water can be initiated to reuse it in a cyclical process.

In another alternate embodiment of the present invention, the system may be developed as a standalone system or may be developed to fit on different types of electrical appliances, such as but not limited to fan, evaporative air coolers, air conditioners, air purifiers. In such systems, reuse of water can be facilitated. In another aspect of the present invention, the system may be embodied to be used in bathroom appliances such as shower, water-tap etc. In yet another embodiment, the system configured as an indoor decontamination system can be operated manually or automatically for spraying the electrically activated water droplets inside an indoor space.

In yet another aspect of the present invention, the present invention provides method for hand sanitization. The method discloses a chemical free hand sanitization method based on the electrically activated water. Further, it also provides a method for full body sanitization of human beings and maintaining the contamination free surroundings around them. Being a harmful chemical free method, this invention may also be used for continuous control of disinfection and infection.

In yet another aspect of the present invention, the method of the present invention may be used for an integrated sterilization system for healthcare facilities for sterilization of hospital supplies, water used in hospital, surface cleaning water, disinfection of air inside hospital to prevent transmission of infection.

In another aspect of the present invention, the sterilization method for preparing disinfectant may be used for inactivation of microbes on food materials to increase their shelf-life and to help in their transport without decay of food.

In yet another aspect of the present invention, the sterilization method for preparing disinfectant may be used for treatment of waste-water and converting waste-water into sterilization substance.

In yet another aspect of the present invention, the sterilization method may be used for air purification applications and for maintaining optimal Indoor Air Quality (IAQ).

While this invention has been described in connection with what is presently considered to be the most practical and preferred embodiment, it is to be understood that the invention is not limited to the disclosed embodiments, but, on the contrary, is intended to cover various modifications and equivalent arrangements included within the scope of the appended claims.

1. A method of sterilization to remove microbial load comprising the steps of:
i. electrolyzing/hydrolyzing water in an electrolysis chamber in presence or absence of at least one electrolyte to produce mixture of gases through a pair of electrodes with a power source;
ii. mixing gases obtained in step (i) with air or water mist through a first fan mounted on the electrolysis chamber, so as to obtain a gas mixture;
iii. exposing the gas mixture obtained in step (ii) to an electric field generated through the first pair of electrodes connected with a power source, said electric field is through any one of: a direct current (DC) voltage of more than 1V; or an alternate current (AC) supply of more than 1 V, 1 hz, said electric field generates in any one of: the electrolysis chamber; the charging chamber, resulting into conversion of gases into ionized species and/or generating other electrically active species (such as radicals), wherein the ionized gas mixture or activated mixture can be used for sterilization of surfaces or air;
iv. dissolving the activated mixture obtained in step (iii) in water to obtain activated water, thereby sterilizing the water;
v. converting the activated water obtained in step (iv) into water mist by breaking the activated water into small water droplets through any one of: a first ultrasonic transducer, in the electrolysis chamber; or through a second ultrasonic sensor in charging chamber;
vi. mixing the water droplets obtained in step (v) with air through any one of: the first fan in the electrolysis chamber; or a second fan mounted in the charging chamber, producing water mist;
vii. passing the water mist obtained in step (vi) through a second pair of electrodes connected with a power source having electric field generated by any one of: the DC voltage of more than 1V; or the AC supply of more than 1 V, 1 hz, so as to gain charge/ion for further activation;
viii. forming electrical species/ions in air and water droplets in the presence of electric field;
ix. transferring the electrical species generated in step (vii) to water droplet, allowing the water droplets to gain charge;
x. breaking micro-droplets of water into nano-droplets through electron clouds or electrostatic repulsion within the droplet due to high charge density, wherein the power source and the second pair of electrodes are configured to break micro-droplets of water into nano-droplets so as to create electrically activated water mist;
xi. dispersing the electrically activated nano-droplets obtained in step (x) in air or transporting the nano-droplets in space away from the site of generation of said activated mist for continuous disinfection of air.

2. The method as claimed in claim 1, wherein method for preparing electrically activated water comprises:
(i) electrolyzing/hydrolyzing water in the electrolysis chamber in presence or absence of at least one electrolyte to produce mixture of gases through the pair of electrodes with the power source;
(ii) mixing gases obtained in step (i) with water mist to obtain a gas mixture;
(iii) exposing the mixture obtained in step (ii) to an electric field generated through the power source and the electrodes, generally high voltage or high power electric field using any one of: the direct current (DC) voltage of more than 1V; or the alternate current (AC) supply of more than 1 V, 1 hz, in any one of: the electrolysis chamber; or the charging chamber, thereby converting gases into ionized species and/or generating other electrically active species (such as radicals);
(iv) dissolving the activated mixture obtained in step (iii) in water to obtain activated water by bubbling it through the water in a repeated process;
(v) breaking the bubbles smaller and increasing their residence time in water to increase their dissolution;
(vi) dispersing electrically activated water on site of generation/surface for sterilization of an object.

3. The method as claimed in claim 1, wherein method for preparing electrically activated water mist comprises:
(i) mixing the activated water droplets obtained from the activated water with air so as to produce activated water mist;
(ii) passing the activated water mist through high power electric field to gain charge/ion for further activation, wherein electric field is generated through any one of: the DC voltage of more than 1V; or the AC supply of more than 1 V, 1 hz
(iii) forming electrical ions in air and water droplets in the presence of electric field;
(iv) transferring the electrical species generated in step (iii) to water droplet, allowing the water droplets to gain charge;
(v) breaking micro-droplets of water into nano-droplets through/by electrostatic repulsion of droplets generated by increased charged density in water droplet;
(vi) dispersing highly activated nano-droplets obtained in step (v) in air or transporting the nano-droplets in space away from the site of generation of said activated mist for continuous disinfection of air.
(vii) generating protective air envelop around the activated water mist stream or air bubbles to protect the mist from getting in contact with surfaces of transporting support such as duct, during the transport.

4. The method as claimed in claim 1, wherein electrolysis of water takes place by maintaining voltage through a power source, such as but not limited to a battery.

5. The method as claimed in claim 1, wherein at least one water pump is configured to connect the charging chamber to the electrolysis chamber for enabling circulation of fluids between the electrolysis chamber and the charging chamber.

6. The method as claimed in claim 1, wherein the activated water may directly be used for surface disinfection or may be converted into water mist by shearing water into smaller droplets like micro-droplets by a dispersing unit, such as but not limited to nozzles or ultrasonic probes or any spraying unit.

7. The method as claimed in claim 1, wherein the activated water droplets dispersed through the dispersing unit are configured to be mixed with air to produce water mist which is subjected to high voltage or high power electric field, generated by any one of: the DC supply; or AC supply or electron clouds or ionized space to gain charge or ions for further activation.

8. The method as claimed in claim 3, the mean aerodynamic size of the water droplets in the electrically activated water mist may be modulated/controlled to match the aerodynamic size to the microbes so as to inactivate the microbes, and wherein water droplets with different aerodynamic sizes may be generated for inactivation of different types of microbes with different aerodynamic sizes.

9. The method as claimed in claim 1, wherein the method can be used for sterilization of but not limited to air, liquid water and solid surfaces.

10. The method as claimed in claim 1, wherein the electrically activated water can be converted into electrically activated mist to disinfect air and surfaces which can be remixed in the water so as to increase charge density or electrical activation of the water.

11. The method as claimed in claim 3, the activated mist may be returned to any one of: the electrolysis chamber; the charging chamber after disinfection of air and may be precipitated through electrically charged plates.

12. A system for obtaining water mist comprising:
an electrolysis chamber having a pair of electrodes and a power source for electrolysis of water;
a first ultrasonic transducer connected to the electrolysis chamber;
a first fan mounted on the electrolysis chamber;
a first pair of electrodes connected to power source for generation of electric field
a charging chamber for receiving electrically activated water from the electrolysis chamber and for generation of electrically activated water mist;
a second fan mounted on the charging chamber;
a second ultrasonic transducer connected to the charging chamber;
at least one water pump configured to connect the charging chamber to the electrolysis chamber;
a pair of second electrodes with a power source connected to the charging chamber;
wherein,
I. the pair of electrodes is configured to obtain a plurality of gases through electrolysis;
II. the first ultrasonic transducer is configured to break water provided in the electrolysis chamber into plurality of droplets;
III. the first fan is configured to mix the plurality of gases with air or water mist and recirculate said gas mixture in the electrolysis chamber, and wherein said mixture is passed through the water for dissolving the activated mixture in water;
IV. the first pair of electrodes with power source exposes said gas mixture to the electric field;
V. the water pump for circulation of fluids between the electrolysis chamber and the charging chamber;
VI. the second ultrasonic transducer configured to break activated water into smaller droplets;
VII. the second fan mounted to the charging chamber is configured to mix air with water droplets so as to generate activated mist; and
VIII. the second pair of electrodes with power source is configured to generate electric field to break water droplet into ultrafine droplets so as to create electrically activated water mist.
